(19) United States
(12) Patent Application Publication
(10) Pub. No.: US 2007/0287718 A1
(43) Pub. Date: Dec. 13, 2007

(54) Methods for the Treatment of Multiple Myeloma Using Roscovitine

(76) Inventors: Simon Richard Green, Dundee (GB); David Maccallum, Angus (GB)

Correspondence Address: LAHIVE & COCKFIELD, LLP, ONE POST OFFICE SQUARE, BOSTON, MA, 02109-2127, US

(21) Appl. No.: 10/578,274

(22) PCT Filed: Nov. 8, 2004
(86) PCT No.: PCT/GB04/04704

§ 371 Date: Apr. 30, 2007

(30) Foreign Application Priority Data
Nov. 6, 2003 (GB) 0325977.7
Mar. 16, 2004 (GB) 0405907.7

(51) Int. Cl. A61K 31/52; A61P 35/00

(52) U.S. Cl. 514/263.4

(57) ABSTRACT

The present invention relates to the use of roscovitine, or a pharmaceutically acceptable salt thereof, in the preparation of a medicament for treating multiple myeloma.

The present invention relates to the therapeutic uses of the compound 2-[(1-ethyl-2-hydroxyethyl)amino]-6-benzylamine-9-isopropylpurine and pharmaceutically acceptable salts thereof.

### BACKGROUND TO THE INVENTION

The prior art has described several compounds that are capable of regulating the cell cycle by virtue of inhibiting cyclin dependent kinases. These compounds include butyrolactone, flavopiridol and 2-(2-hydroxyethylamino)-6-benzylamino-9-methylpurine (olomoucine). Olomoucine and related compounds have been shown to be inhibitors of cdc2. cdc2 (also known as cdk1) is a catalytic sub-unit of a family of cyclin dependent kinases that are involved in cell cycle regulation.

These kinases comprise at least two sub-units, namely a catalytic sub-unit (of which cdc2 is the prototype) and a regulatory sub-unit (cyclin). The cdks are regulated by transitory association with a member of the cyclin family: cyclin A (cdc2, CDK2), cyclin B1-B3 (cdc2), cyclin C (CDK8), cycline D1-D3 (CDK2-CDK4- CDK5-CDK6), cyclin E (CDK2), cyclin H (CDK7).

Each of these complexes is involved in a phase of the cellular cycle. CDK activity is regulated by post-translatory modification, by transitory associations with other proteins and by modifications of their intra-cellular localization. The CDK regulators comprise activators (cyclins, CDK7/cyclin H, cdc25 phosphateses), the p9.sup.CKS and p15.sup.CDK-BP sub-units, and the inhibiting proteins (p16.sup.INK4A, p15.sup.INK4B, p21.sup.Cip1, p18, p27.sup.Kip1).

There is now considerable support in the literature for the hypothesis that CDKs and their regulatory proteins play a significant role in the development of human tumours. Thus, in numerous tumours a temporal abnormal expression of cyclin-dependent kinases, and a major de-regulation of protein inhibitors (mutations, deletions) has been observed.

Roscovitine is the compound 6-benzylamino-2-[(R)-1-ethyl-2-hydroxyethylamino]-9-isopropylpurine. It induces apoptosis from all phases of the cell cycle in tumour cell lines and reduces tumour growth in human tumour xenongrafts in nude mice. The compound is currently in development as an anti-cancer agent.

Roscovitine has been demonstrated to be a potent inhibitor of cyclin dependent kinase enzymes, competing with ATP for its binding site on the kinase. It exhibits greatest in vitro activity against CDK2/cyclin E (Kᵢ=0.12 μM), CDK7/cyclin H (Kᵢ=0.21 μM) and CDK9/cyclin T (Kᵢ=0.39 μM).

CDK inhibitors are understood to block passage of cells from the G1/S and the G2/M phase of the cell cycle. Roscovitine has also been shown to be an inhibitor of retinoblastoma phosphorylation and therefore implicated as acting more potently on Rb positive tumours.

It has now been observed that roscovitine has therapeutic applications in the treatment of certain proliferative disorders that have to date been particularly difficult to treat.

### STATEMENT OF INVENTION

A first aspect of the invention relates to the use of roscovitine, or a pharmaceutically acceptable salt thereof, in the preparation of a medicament for treating mature B-cell malignancies, for example, multiple myeloma.

A second aspect of the invention relates to a method of treating a patient suffering from multiple myeloma comprising administering a therapeutically effective amount of roscovitine or a pharmaceutically effective salt thereof.

A third aspect of the invention relates to a pharmaceutical composition comprising (i) roscovitine, or a pharmaceutically acceptable salt thereof; and optionally (ii) a pharmaceutically acceptable carrier, diluent or excipient, for use in the treatment of multiple myeloma.

A fourth aspect of the invention relates to a method of down regulating expression of an anti-apoptotic gene in multiple myeloma cells, the method comprising contacting the cells with roscovitine, or a pharmaceutically acceptable salt thereof.

A fifth aspect of the invention relates to a method of treating multiple myeloma in a subject, the method comprising administering roscovitine, or a pharmaceutically acceptable salt thereof, to the subject in an amount sufficient to down regulate the expression of an anti-apoptotic gene in the subject.

A sixth aspect of the invention relates to a method of down-regulating Mcl-1 expression in multiple myeloma cells, said method comprising contacting said cells with roscovitine, or a pharmaceutically acceptable salt thereof.

A seventh aspect of the invention relates to a method of treating multiple myeloma in a subject, said method comprising administering roscovitine, or a pharmaceutically acceptable salt thereof, to the subject in an amount sufficient to down-regulate the expression of Mcl-1 in said subject.

An eighth aspect of the invention relates to the use of roscovitine, or a pharmaceutically acceptable salt thereof, in the preparation of a medicament for treating multiple myeloma, wherein the roscovitine or a pharmaceutically acceptable salt thereof, is in an amount sufficient to down-regulate the expression of Mcl-1.

### DETAILED DESCRIPTION

As mentioned above, the present invention relates to the use of roscovitine in the treatment of multiple myeloma.

Roscovitine or 2-[(1-ethyl-2-hydroxyethyl)amino]-6-benzylamine-9-isopropylpurine, is also described as 2-(1-D,L-hydroxymethylpropylamino)-6-benzylamine-9-isopropylpurine. As used herein, the term “roscovitine” encompasses the resolved R and S enantiomers, mixtures thereof, and the racemate thereof.

The in vitro activity of roscovitine is as follows:

| Kinase | IC₅₀ (μM) |
|---|---|
| Cdk1/cyclin B | 2.7 |
| Cdk2/cyclin A | 0.7 |
| Cdk2/cyclin E | 0.1 |
| Cdk7/cyclin H | 0.5 |
| Cdk9/cyclin T1 | 0.8 |
| Cdk4/cyclin D1 | 14.2 |
| ERK-2 | 1.2 |
| PKA | >50 |
| PKC | >50 |

Although the use of roscovitine as an antiproliferative agent is known in the art, to date, there has been no suggestion that it would be effective in the treatment of multiple myeloma, which is known to be particularly difficult to treat and is often resistant to conventional treatments.

Therapeutic Activity

Multiple myeloma (also known as myeloma or plasma cell myeloma) is a hematologic cancer which remains particularly difficult to treat. Specifically, multiple myeloma is a cancer of the plasma cell, an important part of the immune system that produces immunoglobulins to fight infection and disease. Typically, the disease is characterised by marrow plasmacytosis (plasma cell tumours) and overproduction of an intact monoclonal immunoglobulin (IgG, IgA, IgD or IgE) or Bence Jones protein (free monoclonal κ or λ light chains). Multiple myeloma is often associated with multiple osteolytic lesions, hypercalcemia, anemia, renal damage and increased susceptibility to bacterial infections; production of normal immunoglobulin is impaired (The Merck Manual, 17^{th} Edition, page 965). The following information was obtained from www.multiplemyeloma.org.

Cells destined to become immune cells, like all blood cells, arise in the bone marrow from stem cells. Some stem cells develop into the small white blood cells called lymphocytes. The two major classes of lymphocytes are B cells (B lymphocytes) and T cells (T lymphocytes).

Normally, plasma cells develop from B cells when foreign substances (antigens), such as bacteria, enter the body. Plasma cells produce proteins called immunoglobulins (Ig), (antibodies), that help fight disease and infection. Each plasma cell develops in response to a particular foreign substance within the body and produces immunoglobulins specific to that substance. However, when B cells are damaged, the resulting plasma cells become malignant and continue to divide unchecked, thereby generating more malignant plasma cells. These myeloma cells then travel through the bloodstream and collect in the bone marrow, where they cause tissue damage.

Normally, plasma cells make up a very small portion (less than 5%) of cells in the bone marrow. Myeloma cells, however, have adhesion molecules on their surface allowing them to target bone marrow. After they enter the bone marrow, these adhesion molecules allow them to attach to structural cells called stromal cells. Once myeloma cells attach to bone marrow stromal cells, several interactions cause myeloma cells to grow. Firstly, chemical messengers called cytokines are produced by both myeloma cells and stromal cells. These cytokines, such as interleulin 6 (IL-6), stimulate the growth of myeloma cells and inhibit apoptosis. Secondly, myeloma cells produce growth factors that promote the creation of new blood vessels (angiogenesis). These new blood vessels provide the oxygen and nutrients necessary for tumour growth. A growth factor called vascular endothelial growth factor (VEGF) plays a key role in angiogenesis. Angiogenesis helps the myeloma cells increase in number and begin to infiltrate the bone marrow, eventually comprising greater than 10% of the cells present. Finally, mature myeloma cells may fail to activate the immune system and may produce substances that decrease the body's normal immune response to a foreign body, thereby allowing the cells to grow unchecked.

As tumours grow, they invade the hard, outer part of the bone, the solid tissue. In most cases, the myeloma cells spread into the cavities of all the large bones of the body, forming multiple small lesions (hence the name “multiple” myeloma). In some cases, however, the myeloma cells collect in a single bone and form a tumour called a plasmacytoma.

Myeloma cells are identical and produce the same immunoglobulin protein, called monoclonal (M) protein or paraprotein, in large quantities. Although the specific M protein varies from patient to patient, it is always exactly the same in any one patient. These M proteins show up as a “spike” during electrophoresis. Unlike normal immunoglobulin, M protein is of no benefit to the body. Instead, it crowds out normal, functional immunoglobulins. Moreover, levels of functional immunoglobulin are depressed in individuals with myeloma. Although not completely understood, it appears that the functional immunoglobulin made by existing normal plasma cells breaks down more quickly in patients with myeloma than in healthy individuals.

A patient's myeloma is often referred to by the type of immunoglobulin or light chain (kappa or lambda type) produced by the cancerous plasma cell. The frequency of the various immunoglobulin types of myeloma parallels the normal serum concentrations of the immunoglobulins. The most common myeloma types are IgG and IgA. IgG myeloma accounts for about 60% to 70% of all cases of myeloma and IgA accounts for about 20% of cases. Few cases of IgD and IgE myeloma have been reported (The Merck Manual, 17^{th} Edition, page 966).

Although a high level of M protein in the blood is a hallmark of myeloma disease, about 15% to 20% of patients with myeloma produce incomplete immunoglobulins, containing only the light chain portion of the immunoglobulin (also known as Bence Jones proteins). These patients are said to have light chain myeloma, or Bence Jones myeloma. In these patients, M protein is found primarily in the urine, rather than in the blood. These Bence Jones proteins may form deposits in the kidney which block the kidney tubules and can eventually cause kidney damage and subsequent kidney failure. A rare form of myeloma called nonsecretory myeloma affects about 1% of myeloma patients. In this form of the disease, plasma cells do not produce M protein or light chains.

To date, most myeloma patients are treated initially with standard-dose conventional chemotherapy. Patients may receive conventional chemotherapy as their only therapy, or in preparation for high-dose chemotherapy and stem cell transplant. Conventional chemotherapy may also be used prior to a stem cell transplant (“induction therapy”) in order to reduce the tumour burden prior to transplant.

The most common chemotherapeutic agent used in the treatment of myeloma is melphalan, an alkylating drug which cross-links to DNA and ultimately prevents the cell from dividing. Melphalan is typically given orally in combination with prednisone, a potent corticosteroid drug with antimyeloma activity. However, melphalan is less suitable for use as induction therapy as it can damage stem cells in the bone marrow and reduce the number of cells that can be harvested in preparation for a stem cell transplant. More recently, Velcade® (bortezomib) has received FDA approval for the treatment of multiple myeloma. Corticosteroids are sometimes used as an alternative in myeloma therapy, especially in older patients and those who cannot tolerate chemotherapy. The most commonly used corticosteroid in this instance is dexamethasone.

Dexamethasone can also be used as a form of induction therapy, alone or in combination with other agents. The combination of vincristine, Adriamycin® (doxorubicin), and dexamethasone, also known as VAD, is the most commonly used induction therapy. More recently, the combination of doxorubicin (pegylated liposomal doxorubicin [Doxil®/CAELYX®]), vincristine, and reduced-dose dexamethasone (DVd) has been shown to be effective as induction therapy. This new formulation of doxorubicin provides for a slow release of the drug, thus exposing myeloma cells to the drug for a longer period of time. Substituting this new formulation of doxorubicin for doxorubicin in the VAD regimen and reducing the dose of dexamethasone appears to improve the safety profile and convenience of the treatment regimen without compromising efficacy (Hussein et al. Cancer. 2002;95:2160-2168.) The combination of dexamethasone and thalidomide has also been shown to be effective as induction therapy. In contrast to VAD, which is administered intravenously, combination thalidomide and dexamethasone therapy is an oral regimen. (Rajkumar et al. J. Clin Oncol. 2002;20:4319-4323).

Chemotherapy can be administered at doses higher than that used with conventional chemotherapy (“high-dose chemotherapy”). Though more effective in killing myeloma cells than conventional chemotherapy, high-dose chemotherapy also destroys normal blood-forming cells in the bone marrow and is therefore always administered in conjunction with a stem cell transplant, which replaces these cells. High-dose chemotherapy with stem cell transplantation is often used after a patient receives induction therapy (conventional chemotherapy) to reduce the tumour burden. As this regimen is more intensive than conventional chemotherapy, it is used less frequently in patients over the age of 70, and may not be suitable for patients who have significantly impaired kidney function or performance status, or other coexisting conditions. In addition, high-dose chemotherapy is often associated with side effects such as nausea, vomiting, diarrhea, mouth sores, skin rash, and hair loss. Moreover, as the treatment destroys blood-producing cells in the bone marrow, a patient is susceptible to infection, anemia, and bleeding caused by low blood cell counts until engraftment is complete. Graft-versus-host disease (GVHD) is another serious complication that may arise.

Chemotherapy may also be used as “salvage therapy in patients who have not responded to primary or subsequent therapy or who experience relapsed disease after an initial response to therapy. By way of example, thalidomide, an oral agent, is often used as salvage therapy for myeloma patients alone, or in combination with dexamethasone or melphalan and dexamethasone.

Myeloma patients may also be treated using radiotherapy. For example, solitary tumours in bone or soft tissue (plasmacytomas) can be treated using high doses of local radiation therapy, or larger parts of the body may be exposed to high doses of radiation in order to reduce tumour burden or as salvage therapy. Local low-dose radiation therapy is sometimes used as palliative treatment to relieve uncontrolled pain and/or to help prevent or treat bone fractures or spinal-cord compression. Alternatively, total body irradiation is used in conjunction with high-dose chemotherapy prior to stem cell transplantation in order to help kill myeloma cells in the bone marrow. However, radiation therapy is often associated with a number of adverse side effects such as increased tiredness and increased skin sensitivity. Furthermore, as normal bone marrow cells are sensitive to radiation, blood cell counts may temporarily decline following treatment.

In spite of the above advances, multiple myeloma is still incurable and remains particularly difficult to treat. The prognosis for patients is often poor, with a median survival of 2.5 to 3 years.

The present invention provides an alternative treatment for multiple myeloma which comprises the use of roscovitine. To date, there has been no teaching or suggestion in the prior art that roscovitine would be suitable for treating this particular disorder.

Experiments have demonstrated that roscovitine can inhibit the transcription of a wide range of RNA polymerase II transcripts (Lam et al., 2001, Genome Biology 2(10) RESEARCH0041). Furthermore, roscovitine can disrupt nucleolar structure also indicative of a transcription inhibitor. Consistent with these results, roscovitine inhibits CDK7/CyclinH/Mat 1 and CDK 9/Cyclin T in vitro at low μM concentrations (McClue et al., 2002, Int. J. Cancer. 102(5):463-8). These kinases are required for phosphorylation of the heptapeptide repeats in the Carboxy-terminal domain (CTD) of RNA polymerase II resulting in the activation of transcriptional elongation. Inhibition of transcription exerts greatest effect on gene products where both the mRNA and protein have short half lives resulting in rapid decline of the protein levels. A number of key proteins that regulate apoptosis fit into this category, most notably Mcl-1, with a half-life of 30 minutes. Mcl-1 is a protein with homology to Bc1-2 that can inhibit apoptosis by blocking the activity of pro-apoptotic members of the Bc1-2 family such as Bax. Cell survival is regulated by this delicate balance between pro- and anti-apoptotic factors. Mcl-1 has been shown to play a critical role in the survival of a number of cancer cell types including Multiple Myeloma and B cell chronic lymphocytic leukaemia (Bannerman et al., 2001, J Biol Chem. 276 (18):14924-32; Derenne et al., 2002, Blood. 100(1):194-9; Liu et al., 2002, J. Exp. Med. 194(2):113-26; Pederson et al., 2002, Blood. 100(5):1795-801; Zhang et al., 2002, Blood. 99(6):1885-93). Specific reduction in Mcl-1 levels induce apoptosis in these cell types.

In vitro studies on myeloma cells have shown that roscovitine induces rapid dephosphorylation of the carboxy-terminal domain (CTD) of RNA polymerase II. Phosphorylation at these sites is crucial for the role of RNA polymerase II in transcriptional elongation. Treatment of multiple myeloma cells with roscovitine caused a rapid down regulation of Mcl-1 that corresponded with induction of apoptosis determined by TUNEL and PARP cleavage, suggesting that roscovitine may be useful for treating multiple myeloma as well as other malignancies that have defects in the apoptosis pathway. Thus, it is believed that roscovitine causes myeloma cell death by disrupting the balance from cell survival to apoptosis through inhibition of transcription, and down-regulation of Mcl-1.

In one preferred embodiment of the invention, the multiple myeloma is selected from IgA myeloma, IgG myeloma, IgD myeloma, IgE myeloma, Bence Jones myeloma and non-secretory myeloma.

More preferably, the multiple myeloma is IgA or IgG myeloma.

In one preferred embodiment of the invention, the roscovitine is administered in an amount sufficient to inhibit at least one CDK enzyme.

Preferably, the CDK enzyme is selected from CDK1, CDK2, CDK4, CDK7 and CDK9.

In one particularly preferred embodiment, the CDK enzyme is CDK2.

In another particularly preferred embodiment, the CDK enzyme is selected from CDK7 and CDK9.

In another preferred embodiment, the roscovitine down regulates expression of an anti-apoptotic gene. Preferably, the anti-apoptotic gene is Mcl-1.

Thus, in another preferred embodiment, the roscovitine is administered in an amount sufficient to cause a down-regulation of Mcl-1.

In another preferred embodiment, the roscovitine is administered in an amount sufficient to induce apoptosis.

In one particularly preferred embodiment, the roscovitine or pharmaceutically acceptable salt thereof is administered in the absence of another active agent, i.e. in a monotherapeutic regimen. For this embodiment, the roscovitine or pharmaceutically acceptable salt thereof is administered optionally in combination with one or more pharmaceutically acceptable diluents, excipients and/or carriers, but in the absence of one or more other active agents.

Another embodiment of the invention relates to a method of treating a patient suffering from multiple myeloma, wherein said method consists essentially of administering a therapeutically effective amount of roscovitine or a pharmaceutically effective salt thereof.

Yet another embodiment of the invention relates to a method of treating a patient suffering from multiple myeloma, wherein said method consists of administering a therapeutically effective amount of roscovitine or a pharmaceutically effective salt thereof.

A further aspect of the invention relates to a method of down regulating expression of an anti-apoptotic gene in multiple myeloma, the method consisting essentially of contacting the cells with roscovitine, or a pharmaceutically acceptable salt thereof.

A further aspect of the invention relates to a method of down regulating expression of an anti-apoptotic gene in multiple myeloma cells, the method consisting of contacting the cells with roscovitine, or a pharmaceutically acceptable salt thereof.

A further aspect of the invention relates to a method of treating multiple myeloma in a subject, the method consisting essentially of administering roscovitine, or a pharmaceutically acceptable salt thereof, to the subject in an amount sufficient to down regulate the expression of an anti-apoptotic gene in the subject.

A further aspect of the invention relates to a method of treating multiple myeloma in a subject, the method consisting of administering roscovitine, or a pharmaceutically acceptable salt thereof, to the subject in an amount sufficient to down regulate the expression of an anti-apoptotic gene in the subject.

Yet another aspect of the invention relates to a method of down-regulating Mcl-1 expression in multiple myeloma cells, said method consisting essentially of contacting said cells with roscovitine, or a pharmaceutically acceptable salt thereof.

Another aspect of the invention relates to a method of down-regulating Mcl-1 expression in multiple myeloma cells, said method consisting of contacting said cells with roscovitine, or a pharmaceutically acceptable salt thereof.

Another aspect of the invention relates to a method of treating multiple myeloma in a subject, said method consisting essentially of administering roscovitine, or a pharmaceutically acceptable salt thereof, to the subject in an amount sufficient to down-regulate the expression of Mcl-1 in said subject.

Another aspect of the invention relates to a method of treating multiple myeloma in a subject, said method consisting of administering roscovitine, or a pharmaceutically acceptable salt thereof, to the subject in an amount sufficient to down-regulate the expression of Mcl-1 in said subject.

Another aspect of the invention relates to the use of roscovitine, or a pharmaceutically acceptable salt thereof, in the preparation of a medicament for treating multiple myeloma, wherein the roscovitine is administered in a monotherapeutic regimen.

A further aspect of the invention relates to the use of roscovitine alone, or a pharmaceutically acceptable salt thereof, in the preparation of a medicament for treating multiple myeloma.

Another aspect of the invention relates to the use of roscovitine, or a pharmaceutically acceptable salt thereof, in the preparation of a medicament for treating multiple myeloma, wherein the roscovitine or a pharmaceutically acceptable salt thereof, is in an amount sufficient to down-regulate the expression of Mcl-1, and wherein the roscovitine is administered in a monotherapeutic regimen.

Pharmaceutical Compositions

Although roscovitine, (or a pharmaceutically acceptable salt, ester or pharmaceutically acceptable solvate thereof) can be administered alone, for human therapy it will generally be administered in admixture with a pharmaceutical carrier, excipient or diluent.

Thus, one aspect of the invention relates to a pharmaceutical composition comprising (i) roscovitine, or a pharmaceutically acceptable salt thereof; and optionally (ii) a pharmaceutically acceptable carrier, diluent or excipient, for use in the treatment of multiple myeloma.

Another aspect relates to a pharmaceutical composition consisting essentially of (i) roscovitine, or a pharmaceutically acceptable salt thereof; and optionally (ii) a pharmaceutically acceptable carrier, diluent or excipient, for use in the treatment of multiple myeloma.

Yet another aspect relates to a pharmaceutical composition consisting of (i) roscovitine, or a pharmaceutically acceptable salt thereof; and optionally (ii) a pharmaceutically acceptable carrier, diluent or excipient, for use in the treatment of multiple myeloma.

One preferred embodiment of the invention therefore relates to the administration of roscovitine in combination with a pharmaceutically acceptable excipient, diluent or carrier.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The active agent of the present invention can be present in the form of a salt or an ester, in particular a pharmaceutically acceptable salt or ester.

Pharmaceutically acceptable salts of the active agent of the invention include suitable acid addition or base salts thereof. A review of suitable pharmaceutical salts may be found in Berge et al, J. Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

The invention also includes where appropriate all enantiomers and tautomers of the active agent. The man skilled in the art will recognise compounds that possess optical properties (one or more chiral carbon atoms) or tautomeric characteristics. The corresponding enantiomers and/or tautomers may be isolated/prepared by methods known in the art.

Stereo and Geometric Isomers

The active agent of the invention may exist in the form of different stereoisomers and/or geometric isomers, e.g. it may possess one or more asymmetric and/or geometric centres and so may exist in two or more stereoisomeric and/or geometric forms. The present invention contemplates the use of all the individual stereoisomers and geometric isomers of the agent, and mixtures thereof. The terms used in the claims encompass these forms, provided said forms retain the appropriate functional activity (though not necessarily to the same degree).

The present invention also includes all suitable isotopic variations of the active agent or pharmaceutically acceptable salts thereof An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agents of the present invention and pharmaceutically acceptable salts thereof can generally be prepared by conventional procedures using appropriate isotopic variations of suitable reagents.

Solvates

The present invention also includes solvate forms of the active agent of the present invention. The terms used in the claims encompass these forms.

Polymorphs

The invention furthermore relates to various crystalline forms, polymorphic forms and (an)hydrous forms of the active agent. It is well established within the pharmaceutical industry that chemical compounds may be isolated in any of such forms by slightly varying the method of purification and or isolation form the solvents used in the synthetic preparation of such compounds.

Prodrugs

The invention further includes the active agent of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredients can be incorporated into a cream consisting of an aqueous emulsion of polyethylene glycols or liquid paraffin. The active ingredients can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

In a particularly preferred embodiment, the combination or pharmaceutical composition of the invention is administered intravenously.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a. variety of factors including the activity of the active agent, the metabolic stability and length of action of the agent, the age, body weight, general health, sex, diet, mode and time of administration, rate of excretion, drug combination, the severity of the particular condition, and the individual undergoing therapy. Dosages and frequency of application are typically adapted to the general medical condition of the patient and to the severity of the adverse effects caused, in particular to those caused to the hematopoietic, hepatic and to the renal system. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, or from 2 to 20 mg/kg body weight. More preferably the agent may be administered at a dose of from 0.1 to 1 mg/kg body weight.

As described above, roscovitine is preferably administered in a therapeutically effective amount, preferably in the form of a pharmaceutically acceptable amount. This amount will be familiar to those skilled in the art. By way of guidance, roscovitine is typically administered orally or intravenously at a dosage of from about 0.05 to about 5 g/day, preferably from about 0.5 to about 5 g/day or 1 to about 5 g/day, and even more preferably from about 1 to about 3 g/day. Roscovitine is preferably administered orally in tablets or capsules. The total daily dose of roscovitine can be administered as a single dose or divided into separate dosages administered two, three or four times a day.

Combinations

In one preferred embodiment of the invention, roscovitine is administered in combination with one or more other antiproliferative agents. In such cases, the compounds of the invention may be administered consecutively, simultaneously or sequentially with the one or more other antiproliferative agents.

It is known in the art that many drugs are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining drugs are that it may promote additive or possible synergistic effects through biochemical interactions and also may decrease the emergence of drug resistance which would have been otherwise responsive to initial treatment with a single agent.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery.

The present invention is further illustrated by way of example, and with reference to the following figures, wherein:

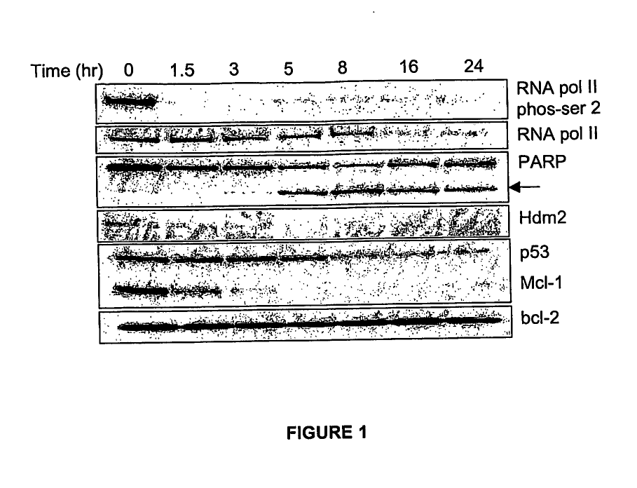

FIG. 1 shows a Western blot for LP-1 multiple myeloma cells treated with 30 μM CYC202.

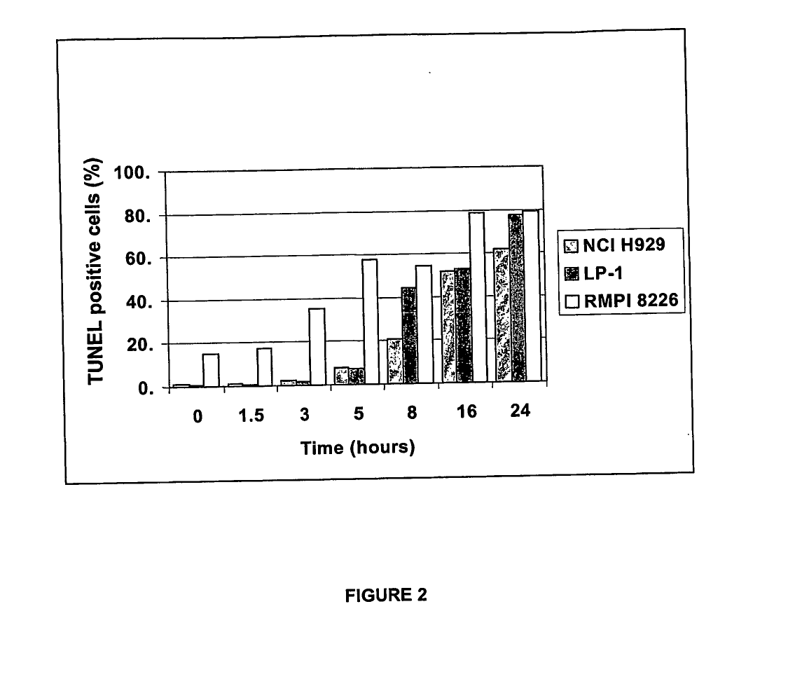

FIG. 2 shows the effect of 30 μM of CYC202 on the induction of apoptosis in multiple myeloma cells measured by TUNEL.

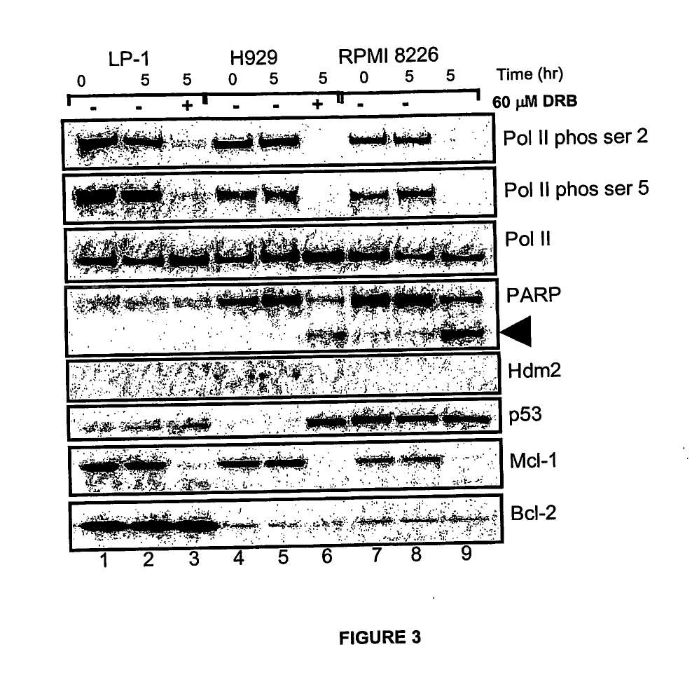

FIG. 3 shows that the transcription inhibitor DRB inhibits phosphorylation of the CTD of RNA polymerase II and down regulates Mcl-1 and Hdm2 levels. Myeloma cells were treated with DMSO or 60 mM DRB, collected at the indicated time points, lysed, separated by SDS PAGE, blotted and probed for a range of antigens. Arrow indicates cleaved form indicative of apoptosis.

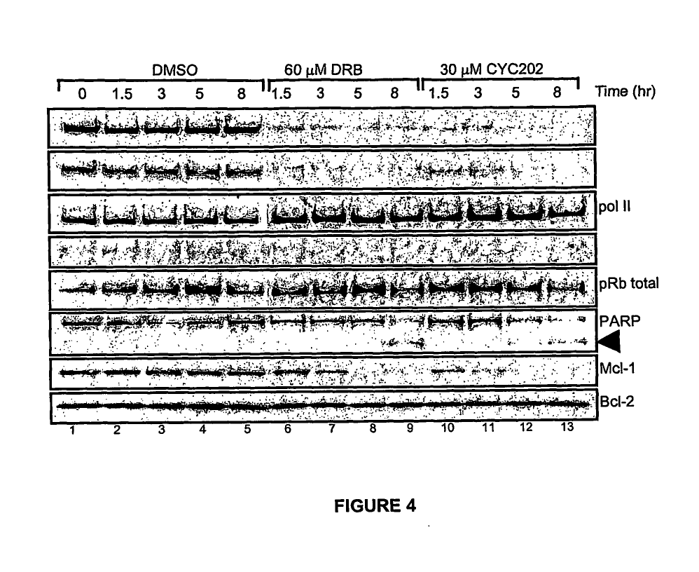

FIG. 4 shows that DRB and CYC202 affect phosphorylation of the C-terminus of RNA polymerase II with similar kinetics. LP-1 cells were treated with DMSO, 60 mM DRB or 30 mM CYC202 collected at the indicated time points lysed, separated by SDS PAGE, blotted and probed for a range of antigens. Arrow indicates cleaved form indicative of apoptosis.

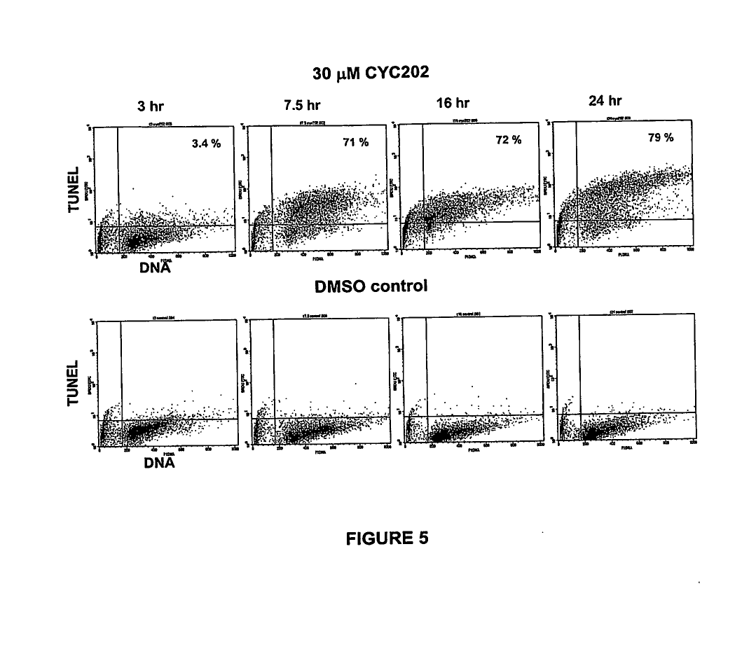

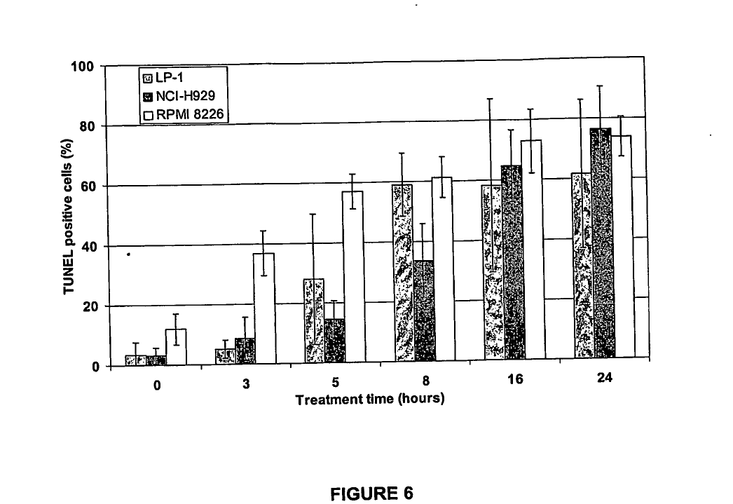

FIGS. 5 and 6 shows that CYC202 induces apoptosis rapidly in myeloma cells determined by TUNEL. Myeloma cells were treated with DMSO or 30 mM CYC202 and collected at the indicated time points, fixed and processed for TUNEL. FIG. 5: representative raw flow cytometry data for LP-1 cells; FIG. 6: averaged data for three experiments in three different myeloma cell lines.

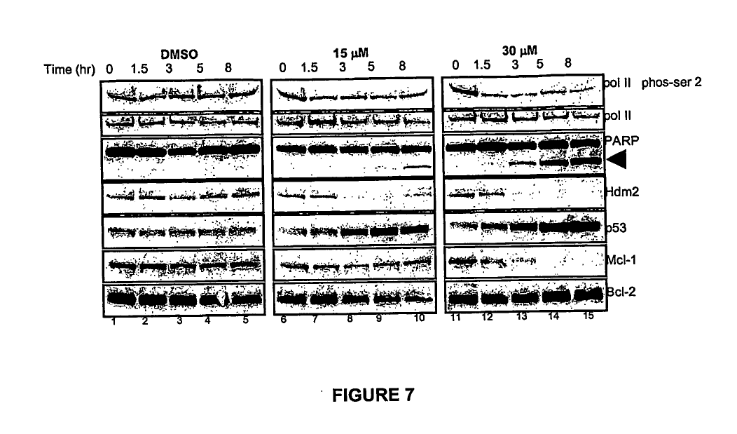

FIG. 7 shows that CYC202 inhibits phosphorylation of the CTD of RNA polymerase II, down regulates Mcl-1 and Hdm2 and induces apoptosis. H929 cells were treated with DMSO, 15 mM or 30 mM CYC202 collected at the indicated time points lysed, separated by SDS PAGE, blotted and probed for a range of antigens. Arrow indicates cleaved form indicative of apoptosis.

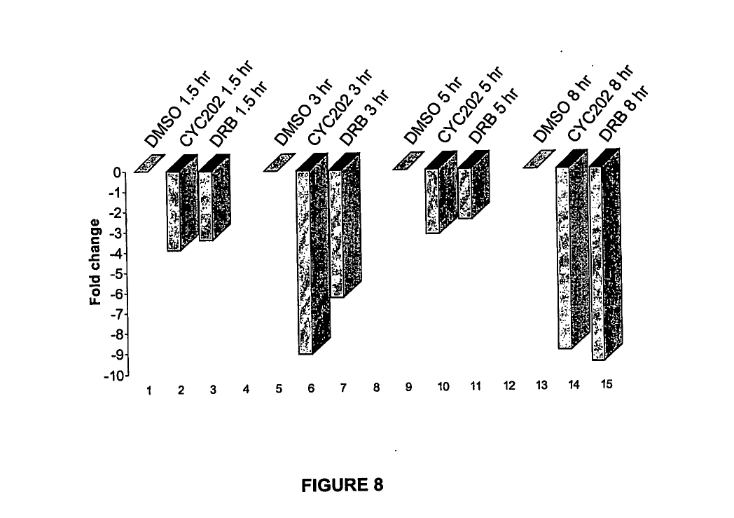

FIG. 8 shows that CYC202 induces down regulation of Mcl-1 mRNA. LP-1 cells were treated with DMSO, 60 mM DRB or 30 mM CYC202, collected at the indicated time points, RNA extracted and levels of Mcl-1 mRNA were determined by real time PCR and normalised to 28S rRNA.

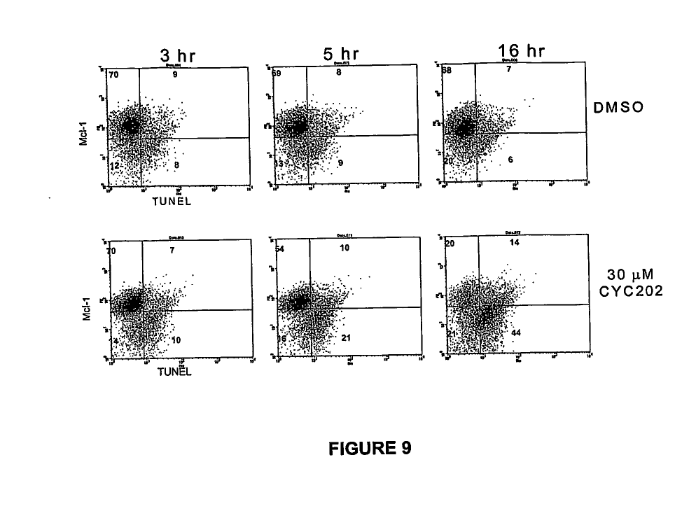

FIG. 9 shows down regulation of Mcl-1 levels coincides with appearance of apoptotic cells. H929 cells were treated with DMSO or 30 mM CYC202 fixed and labelled for TUNEL and Mcl-1 and analysed by flow cytometry.

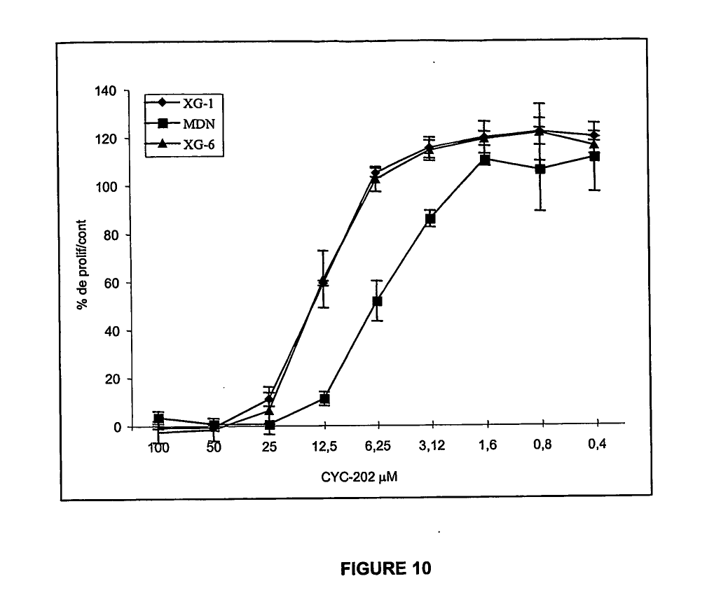

FIG. 10 shows the effect of CYC202 on myeloma cell proliferation using three IL-6 dependent human myeloma cell lines, XG-1, MDN and XG-6 (% of proliferation versus concentration of CYC202).

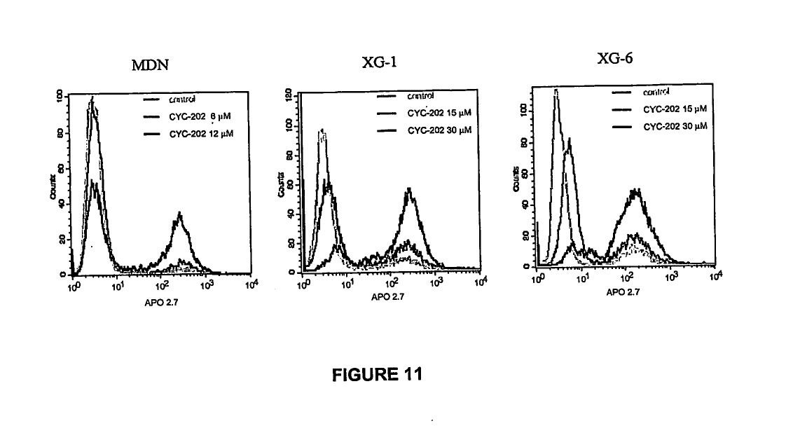

FIG. 11 shows the induction of apoptosis by CYC202 at day 3 as measured by apo2.7 immunostaining. A significant and strong induction of apoptosis was obtained at 12 μM for MDN and at 30 μM for XG1 and XG-6.

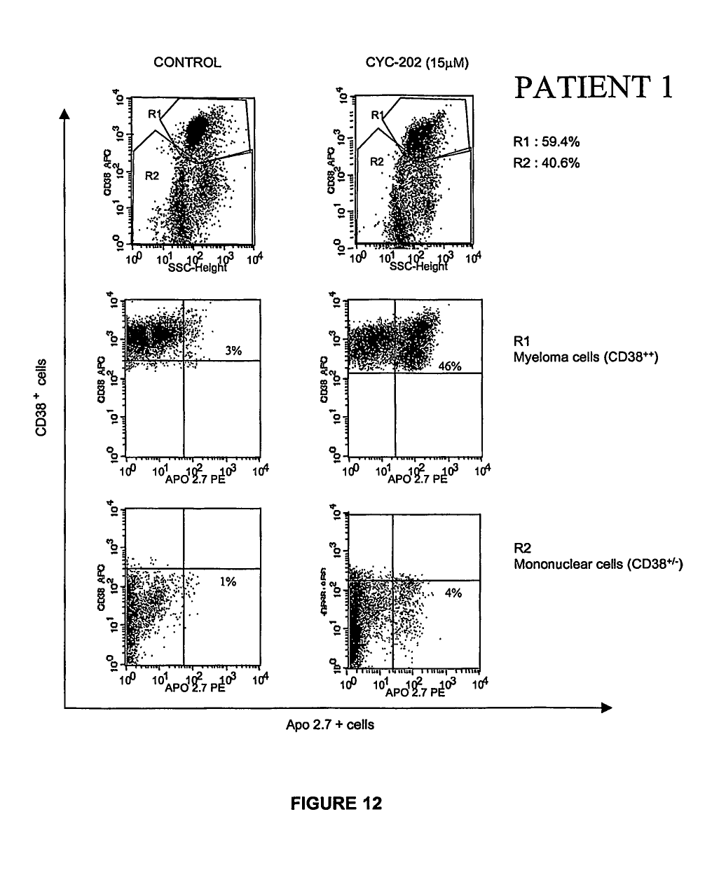

FIG. 12 shows the results of treating cells taken from a multiple myeloma patient (ex vivo) at day 2 with a concentration of 15 μM of CYC202. Myeloma cells were identified by their high expression of CD38.

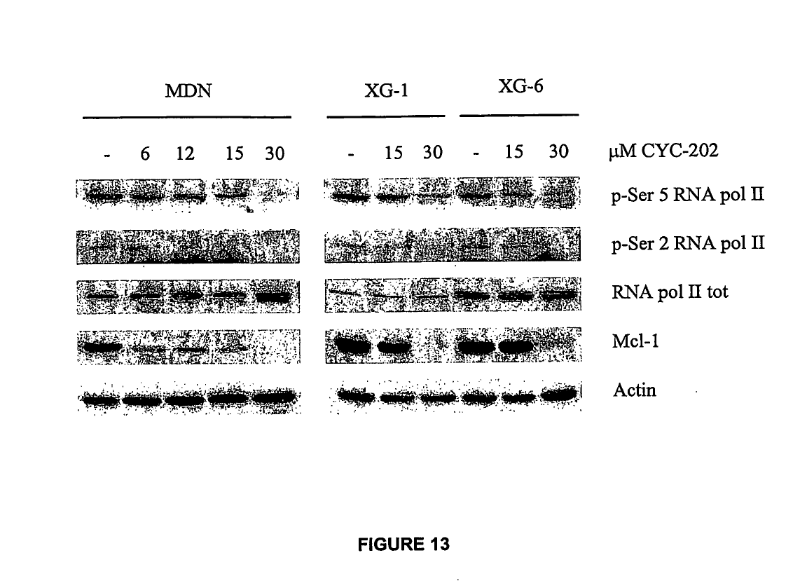

FIG. 13 shows the expression of Mcl-1 and the phosphorylation of RNA pol II in human myeloma cell lines, XG-1, MDN and XG-6, after seven hours treatment with CYC202, as measured by western blotting.

### EXAMPLES

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

Preparation of Roscovitine

Roscovitine was prepared in accordance with the method disclosed in EP0874847B (CNRS). As used herein, the term “CYC202” refers to a single enantiomer of roscovitine, namely, 2-(1-R-hydroxymethylpropylamino)-6-benzylamino-9-isopurine.

In Vitro Activity of CYC202 Against Multiple Myeloma Cells

Studies were carried out to investigate the effect of CYC202 on LP-1, NCI-H929, RPMI 8226 cell lines and OPM-2 and U266 multiple myeloma cell lines. All cell lines were obtained from the DSMZ (Deutsche Sammlung von Mikroorganismen und Zellkulturen).

The effect of CYC202 on molecular events in LP-1 multiple myeloma cells was investigated. LP-1 cells were treated with 30 μM CYC202 and at time points, cells were collected washed and lysed. Whole cell lysates were fractionated by either 3-8% or 10% SDS-PAGE and analysed by Western blotting using antibodies indicated [Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.]. Western blotting was performed for Mcl-1, an antiapoptotic protein [J. Biol. Chem., 274: 1801-1813, 1999; J. Cell Biol., 128(6): 1173-1187, 1995; Proc. Nat. Acad. Sci. USA, 90: 3516-3520, 1993]. Cleavage of PARP [FASEB Journal 10: 587-597, 1996; Science, 267: 1456-1462, 1995; Biochim. Biophys. Acta, 950:147-160, 1988; J. Biol. Chem, 271(9): 4961-4965, 1996; Nature, 371: 346-347, 1994], an indicator of apoptosis, was also determined.

Flow Cytometry Analysis

Cells were seeded onto 90 mm diameter plates. After 24 hr, cells were treated with CYC202 or the equivalent amount of vehicle (DMSO). Cells were harvested at various time points after addition of the drug. After washing once in PBS, cells were fixed in ice-cold 70% v/v ethanol and stored for up to 2 weeks at −20° C. Cells were washed twice in PBS +1% w/v BSA to remove fixative and re-suspended in PBS containing 50 μg/ml propidium iodide and 50 μg/ml RnaseA. After incubation at room temperature for 20 min, cells were analysed using flow cytometry.

A Becton Dickinson LSR flow cytometer was used for these studies, in accordance with the manufacturers recommendations. The argon ion laser set at 488 mn was used as an excitation source. Red fluorescence (575±26 nm) was acquired on a linear scale and pulse width analysis was used to exclude cell doublets and aggregates from the analysis. Cells with a DNA content of between 2 n and 4 n were designated as being in G1, S or G2/M phases of the cell cycle, as defined by the level of red fluorescence. Cells showing less than 2 n DNA content were designated as sub-G1 cells. The number of cells in each cell cycle compartment was expressed as a percentage of the total number of cells present. Analysis of apoptosis was determined using terminal deoxynucleotidyltransferase dUTP nick end labelling (TUNEL) using the APO-DIRECT kit from BD biosciences (catalog number 556381) according to the manufacturer's instructions.

Results

Treatment of multiple myeloma cell lines with 30 μM CYC202 induces rapid apoptosis seen in the majority of cells by eight hours.

Studies showed that the transcription inhibitor DRB inhibits phosphorylation of the CTD of RNA polymerase II and down regulates Mcl-1 and Hdm2 levels (FIG. 3). FIG. 4 indicates that DRB and CYC202 affect phosphorylation of the C-terminus of RNA polymerase II with similar kinetics.

Detailed studies of three cell lines showed that CYC202 induces rapid dephosphorylation of RNA polymerase II at the serine 2 position of the CTD of RNA pol II, by one and a half hours in LP-1 cells (FIGS. 1 and 7). Phosphorylation at this site is crucial for transcriptional elongation of RNA polymerase II derived transcripts. Consistent with this result, the levels of Mcl-1 protein are rapidly reduced in all cell lines, by three hours. Studies confirmed that the levels of Mcl-1 mRNA were reduced as measured by real-time PCR in LP-1 cells (FIG. 8). Levels of other proteins with short half-lives were also reduced including Hdm2 that regulates the p53 pathway. Low levels of Mcl-1 corresponded with induction of apoptosis determined by both PARP cleavage analysed by Western blotting (FIG. 1) and by TUNEL labelling analysed by flow cytometry (FIGS. 2, 5, 6 and 9).

These results suggest that CYC202 kills myeloma cells through the induction of apoptosis by down-regulating the transcription of key genes required for survival of malignant B-cells.

Effect of CYC 202 on Myeloma Cell Proliferation

The effect of CYC202 on the proliferation of three IL-6 dependent human myeloma cell lines (XG1, XG-6 and MDN) was investigated. Cells were cultured in the presence of 3 ng/ml IL-6 with or without CYC202 for two days then proliferation was measured using a MTT proliferation assay. The concentration range tested was from 0.4 μM to 100 μM. The IC50 of proliferation inhibition was 15 μM for XG1 and XG6 and 6 μM for MDN (FIG. 10). A total inhibition of myeloma cell proliferation was observed at 25 μM of CYC202 for the three myeloma cell lines.

Induction of Apoptosis by CYC202

The induction of apoptosis at day 3 by apo2.7 was investigated by immunostaining. A significant and strong induction of apoptosis was obtained at 12 μM for MDN and at 30 μM for XG1 and XG-6 (FIG. 11).

% of apoptotic cells after 3 day treatment

| CYC 202 | XG-1 | XG-6 | MDN |
|---|---|---|---|
| 3 μM | NT | NT | 3% |
| 6 μM | NT | NT | 5% |
| 7.5 μM | 2% | 4% | |
| 12 μM | | | 41% |
| 15 μM | 18% | 15% | |
| 30 μM | 70% | 74% | |

NT non tested

A concentration of 15 μM CYC202 was tested on cells taken from a multiple myeloma patient at day 2 (FIG. 12). Myeloma cells were identified by their high expression of CD38. The number of apoptotic cells in the myeloma cell population was of 46% whereas only 4% of the non myeloma cell population was induced on apoptosis. This result seems to indicate that the myeloma cell population was more sensitive to CYC202 than the non malignant bone marrow cell population.

Modulation of Mcl-1 and RNA pol II Expression After CYC202 Treatment

The expression of MCL-1 and the phosphorylation of RNA pol II was analysed by western blotting after seven hours treatment with CYC202 (FIG. 13). In MDN cells, a decrease of Mcl-1 expression was observed when the cells were treated either with 6 or 15 μM of CYC202, whereas a total downregulation of Mcl-1 was observed with 30 μM of CYC202. The downregulation of Mcl-1 preceded the induction of apoptosis. For XG-1 and XG-6, only a concentration of 30 μM induced a strong downregulation of Mcl-1. A total downregulation of the phosphorylation of RNA polII on Ser-2 was observed for the three cell lines with 30 μM treatment of CYC202.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

1. A method of treating multiple myeloma in a subject, comprising administering roscovitine, or a pharmaceutically acceptable salt thereof, to said subject.

2. The method of claim 1, further comprising administering the roscovitine with a pharmaceutically acceptable carrier, diluent or excipient.

3. The method of claim 1, wherein the roscovitine is administered in an amount sufficient to inhibit at least one CDK enzyme.

4. The method of claim 3, wherein the CDK enzyme is selected from the group consisting of CDK1, CDK2, CDK4, CDK7 and CDK9.

5. The method of claim 3, wherein the CDK enzyme is CDK1 and or CDK2.

6. The method of claim 3, wherein the CDK enzyme is CDK7 or CDK9.

7. The method of claim 1, wherein the multiple myeloma is selected from the group consisting of IgA myeloma, IgG myeloma, IgD myeloma, IgE myeloma, Bence Jones myeloma and non-secretory myeloma.

8. The method of claim 7, wherein the multiple myeloma is IgA or IgG myeloma.

9. The method of claim 1, further comprising administering the roscovitine with at least one additional antiproliferative agent.

10-18. (canceled)

19. A pharmaceutical composition comprising (i) roscovitine, or a pharmaceutically acceptable salt thereof; and optionally (ii) a pharmaceutically acceptable carrier, diluent or excipient, for use in the treatment of multiple myeloma.

20. A method of down regulating expression of an anti-apoptotic gene in multiple myeloma cells, the method comprising contacting the cells with roscovitine, or a pharmaceutically acceptable salt thereof.

21. A method of treating multiple myeloma in a subject, the method comprising administering roscovitine, or a pharmaceutically acceptable salt thereof, to the subject in an amount sufficient to down regulate the expression of an anti-apoptotic gene in the subject.

22. The method of claim 20, wherein the anti-apoptotic gene is Mcl-1.

23. A method of down-regulating Mcl-1 expression in multiple myeloma cells, said method comprising contacting said cells with roscovitine, or a pharmaceutically acceptable salt thereof.

24. A method of treating multiple myeloma in a subject, said method comprising administering roscovitine, or a pharmaceutically acceptable salt thereof, to the subject in an amount sufficient to down-regulate the expression of Mcl-1 in said subject.

25-26. (canceled)

27. The method of claim 21, wherein the anti-apoptotic gene is Mcl-1.
